(19) United States
(12) Patent Application Publication
(10) Pub. No.: US 2019/0167158 A1
(43) Pub. Date: Jun. 6, 2019

(54) INFORMATION PROCESSING APPARATUS

(71) Applicant: FUJI XEROX CO.,LTD., Tokyo (JP)

(72) Inventor: Hideto YUZAWA, Kanagawa (JP)

(73) Assignee: FUJI XEROX CO.,LTD., Tokyo (JP)

(21) Appl. No.: 16/005,690

(22) Filed: Jun. 12, 2018

(30) Foreign Application Priority Data
Dec. 5, 2017 (JP) 2017-233631

(51) Int. Cl. A61B 5/11; A61B 5/0205; G06F 19/00; A61B 5/024

(52) U.S. Cl. CPC A61B 5/1118; A61B 5/024; G06F 19/00; A61B 5/0205

(57) ABSTRACT

An information processing apparatus includes a specifying unit that specifies an activity degree of a participant based on biometric information obtained from a living body of the participant participating in a gathering, an acquisition unit that acquires motion information indicating a motion of the participant, a generation unit that generates correlation information indicating a correlation between the motion information and the activity degree, and a notification unit that performs a notification of state information indicating a state of the gathering by using the correlation information.

### CROSS-REFERENCE TO RELATED APPLICATIONS

This application is based on and claims priority under 35 USC 119 from Japanese Patent Application No. 2017-233631 filed Dec. 5, 2017.

### BACKGROUND

### (i) Technical Field

The present invention relates to an information processing apparatus.

### (ii) Related Art

Some of participants participating in gatherings such as conferences may be in psychological states which are difficult to be understood from their appearance. In particular, in a communication system and the like in which participants in remote areas gather together via a communication line, limited information captured by a camera, a microphone, and the like is used. Thus, recognizing a state of a participant in a remote area has difficulty in comparison to a gathering in which face-to-face communication is performed. A technology of using biometric information obtained from the living body of each participant for managing a gathering has been developed.

### SUMMARY

According to an aspect of the invention, there is provided an information processing apparatus which includes a specifying unit that specifies an activity degree of a participant based on biometric information obtained from a living body of the participant participating in a gathering, an acquisition unit that acquires motion information indicating a motion of the participant, a generation unit that generates correlation information indicating a correlation between the motion information and the activity degree, and a notification unit that performs a notification of state information indicating a state of the gathering by using the correlation information.

### BRIEF DESCRIPTION OF THE DRAWINGS

Exemplary embodiment(s) of the present invention will be described in detail based on the following figures, wherein:

FIG. 1 is a diagram illustrating a configuration of an information processing system 9 according to an exemplary embodiment;

FIG. 2 is a diagram illustrating an example of a configuration of an aggregation device 4;

FIG. 3 is a diagram illustrating an example of a configuration of a terminal 2;

FIG. 4 is a diagram illustrating a configuration of a server device 1;

FIGS. 5A to 5D are diagrams illustrating a database stored in a storage unit 12;

FIG. 6 is a diagram illustrating a functional configuration of the server device 1;

FIG. 7 is a flowchart illustrating a flow of an operation of the server device 1 specifying an activity degree;

FIG. 8 is a flowchart illustrating a flow of an operation of the server device 1 generating a correlation coefficient;

FIG. 9 is a flowchart illustrating a flow of an operation of the server device 1 performing notification of state information of a gathering;

FIG. 10 is a diagram illustrating an example of changes of the activity degree and a motion amount of a participant with time;

FIG. 11 is a diagram illustrating the state information of which notification is performed;

FIG. 12 is a flowchart illustrating a flow of an operation of the server device 1 performing notification of state information of the gathering in a modification example;

FIG. 13 is another flowchart illustrating a flow of an operation of the server device 1 performing notification of state information of the gathering in a modification example;

FIG. 14 is a diagram illustrating an example of changes of the activity degree and the motion amount of a participant with time, in the modification example; and

FIGS. 15A and 15B are diagrams illustrating the state information of which notification is performed in the modification example.

### DETAILED DESCRIPTION

### Exemplary Embodiment

Overall Configuration of Information Processing System

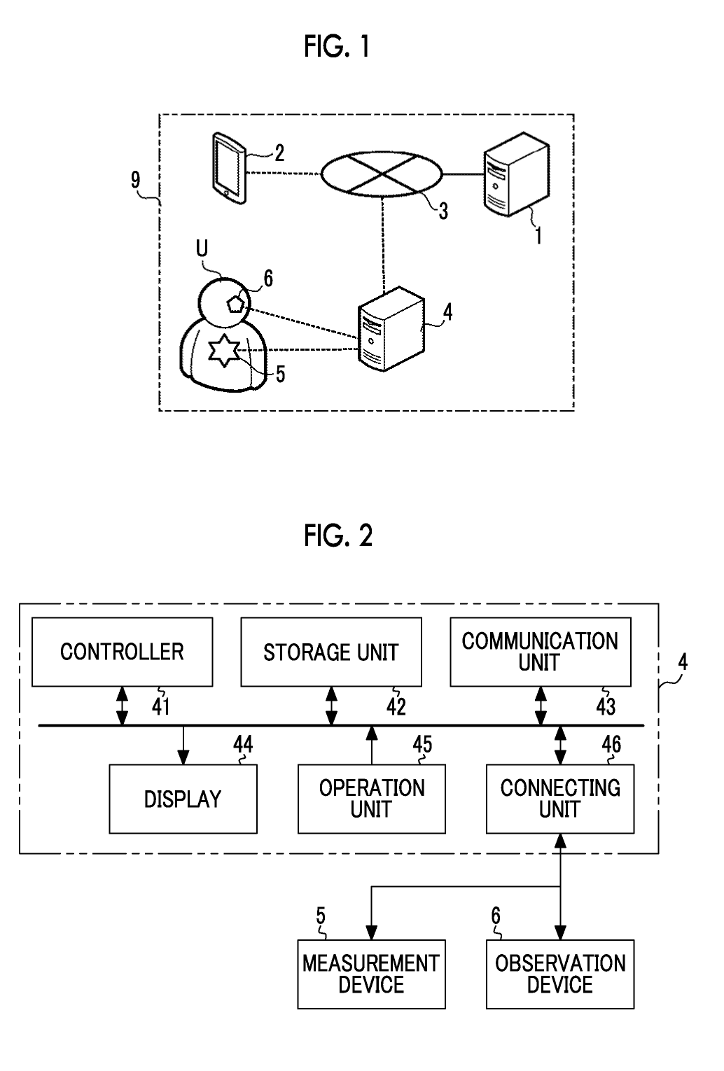

FIG. 1 is a diagram illustrating an information processing system. 9 according to an exemplary embodiment. The information processing system 9 includes a server device 1, a terminal 2, an aggregation device 4, and a communication line 3 which is connected to the server device, the terminal, and the aggregation device so as to enable communication with each other. The information processing system 9 includes a measurement device 5 and an observation device 6. In the information processing system 9 illustrated in FIG. 1, the measurement device 5 and the observation device 6 are connected to the aggregation device 4. The information processing system 9 may further include an imaging device or a sound collection device such that a participant in a gathering recognizes the posture or the sound of another participant.

The information processing system 9 may include plural server devices 1 and plural communication lines 3. In the exemplary embodiment, the communication line 3 links plural venues. The terminal 2, the aggregation device 4, the measurement device 5, and the observation device 6 are provided in each venue. However, for simple descriptions, one terminal, one aggregation device, one measurement device, and one observation device are illustrated in FIG. 1.

The server device 1 is an information processing apparatus that provides information (state information) indicating a state of a gathering based on an activity degree and motion information in the gathering held with the plural venues linked.

Here, “the gathering” means that plural venues are linked by a communication line and participants in each of the venues perform communication with each other in a form of, for example, dialogues and gestures. For example, a conference, a debate, a study group, a social meeting, and a massive multiplayer game are provided as “the gathering”.

The terminal 2 is a terminal device that delivers information of a participant to a user of the information processing system 9 such as a participant or a host of a gathering.

The communication line 3 is a line that connects the server device 1 with the terminal 2 and the aggregation device 4 to enable communication with each other. For example, the Internet is provided.

For example, at least one aggregation device 4 is provided in each venue and the aggregation device aggregates pieces of information of participants U who are in the venue and participate in a gathering. The aggregation device 4 is connected to the measurement device 5 and the observation device 6 so as to aggregate information provided by each of the measurement device and the observation device and to transmit the aggregated information to the server device 1.

In FIG. 1, the aggregation device 4 is connected to one measurement device 5 and one observation device 6. However, the aggregation device may be connected to plural measurement devices or plural observation devices.

The measurement device 5 is a device that measures information such as an electrocardiographic waveform, a pulse wave, and a heartbeat of a participant U, which is obtained from the living body of the participant (referred to as biometric information below). The measurement device 5 includes plural electrodes which are brought into contact with the skin of a participant U, for example. The measurement device measures an electrocardiographic waveform by measuring impedance between the electrodes.

The observation device 6 is a device that observes information regarding a motion (motion information), such as the amount of a motion (motion amount) of the participant U, a direction of the motion, a portion of the participant U, which is moving, and the like, by using an acceleration sensor mounted on, for example, the head of the participant U or the arms and legs thereof. The observation device 6 may observe the motion amount of each moving portion. For example, in a case where the position of the heart of a participant moves with movement of the body thereof, observation may be performed in a manner that a component along the gravity is separated among moving directions and the separated component is used as the motion amount.

Configuration of Aggregation Device

FIG. 2 is a diagram illustrating an example of a configuration of the aggregation device 4. The aggregation device 4 includes a controller 41, a storage unit 42, a communication unit 43, a display 44, an operation unit 45, and a connecting unit 46.

The controller 41 includes a central processing unit (CPU), a read only memory (ROM), and a random access memory (RAM). The controller controls the units of the aggregation device 4 by the CPU reading and executing a computer program (simply referred to as a program below) stored in the ROM and the storage unit 42.

The communication unit 43 is a communication circuit connected to the communication line 3 in a wired or wireless manner. The aggregation device 4 transmits and receives information to and from the server device 1 connected to the communication line 3, by the communication unit 43.

The operation unit 45 includes operation components such as operation buttons, a keyboard, and a touch panel for inputting various instructions. The operation unit receives an operation by a user and transmits a signal corresponding to operation contents, to the controller 41.

The display 44 includes a display screen such as a liquid crystal display. The display displays an image under a control of the controller 41. A transparent touch panel of the operation unit 45 may be disposed on the display screen to overlap the display screen.

The aggregation device 4 may not include the operation unit 45 or the display 44.

The storage unit 42 is a large capacity storage section such as a solid state drive and a hard disk drive. The storage unit stores various programs, pieces of data, and the like which are to be read out by the CPU of the controller 41.

The connecting unit 46 is an interface for performing connection to a device that supplies various types of information. The connecting unit 46 illustrated in FIG. 2 is connected to the measurement device 5 and the observation device 6. The connecting unit 46 may be connected to an imaging device or a sound collection device (not illustrated) and may supply data generated by the imaging device or the sound collection device to the controller 41.

The connecting unit 46 acquires biometric information such as an electrocardiographic waveform of a participant U, from the measurement device 5 and acquires motion information of the participant U from the observation device 6.

Various types of information acquired through the connecting unit 46 are supplied to the controller 41 and are associated with a time point and the participant U under a control of the controller 41. The resultant is transmitted to the server device 1 through the communication unit 43 and the communication line 3.

Configuration of Terminal

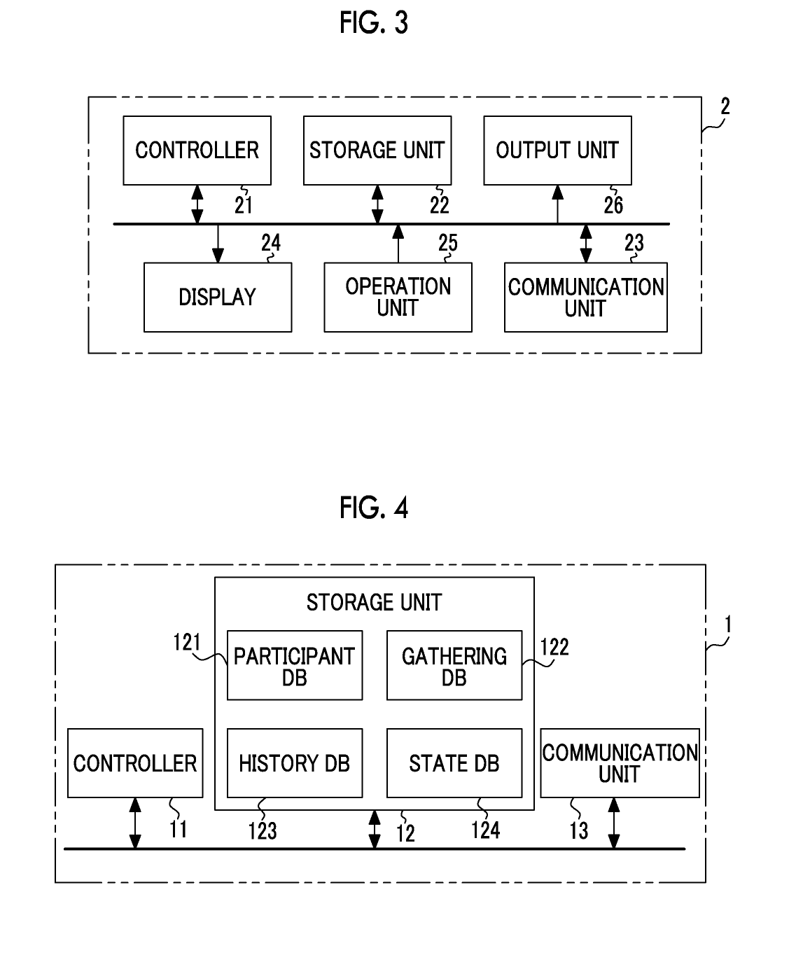

FIG. 3 is a diagram illustrating an example of a configuration of the terminal 2. The terminal 2 includes a controller 21, a storage unit 22, a communication unit 23, a display 24, an operation unit 25, and an output unit 26.

The controller 21 includes a CPU, a ROM, and a RAM. The controller 21 controls the units of the terminal 2 by the CPU reading and executing a program stored in the ROM and the storage unit 22.

The communication unit 23 is a communication circuit connected to the communication line 3 in a wired or wireless manner. The terminal 2 transmits and receives information to and from the server device 1 connected to the communication line 3, by the communication unit 23.

The operation unit 25 includes operation components such as operation buttons, a keyboard, and a touch panel for inputting various instructions. The operation unit receives an operation by a user and transmits a signal corresponding to operation contents, to the controller 21. The terminal 2 may not include the operation unit 25.

The display 24 includes a display screen such as a liquid crystal display. The display displays an image under a control of the controller 21. A transparent touch panel of the operation unit 25 may be disposed on the display screen to overlap the display screen. The terminal 2 may not include the display 24.

The storage unit 22 is a large capacity storage section such as a solid state drive and a hard disk drive. The storage unit stores various programs, pieces of data, and the like which are to be read out by the CPU of the controller 21.

The output unit 26 is a device that outputs information detected by various senses (auditory sense, tactile sense, and the like) of a user, under control of the controller 21. For example, the output unit 26 is a speaker that outputs sound represented by sound data, or a vibration generator that comes into contact with the body of the user and transmits vibration.

In a case where the output unit 26 is a vibration generator, the terminal 2 may be a pointer that points a position on a screen on which an image is displayed, for example. In this case, information is transferred to a user who holds the terminal 2 with the hand, by the output unit 26 vibrating.

Configuration of Server Device

FIG. 4 is a diagram illustrating a configuration of the server device 1. The server device 1 includes a controller 11, a storage unit 12, and a communication unit 13.

The controller 11 includes a CPU, a ROM, and a RAM. The controller 11 controls the units of the server device 1 by the CPU reading and executing a program stored in the ROM and the storage unit 12.

The communication unit 13 is a communication circuit connected to the communication line 3 in a wired or wireless manner. The server device 1 transmits and receives information to and from the terminal 2 and the aggregation device 4 which are connected to the communication line 3, by the communication unit 13.

The storage unit 12 is a large capacity storage section such as a hard disk drive. The storage unit stores various programs, pieces of data, and the like which are to be read out by the CPU of the controller 11.

The storage unit 12 stores a participant DB 121, a gathering DB 122, a history DB 123, and a state DB 124.

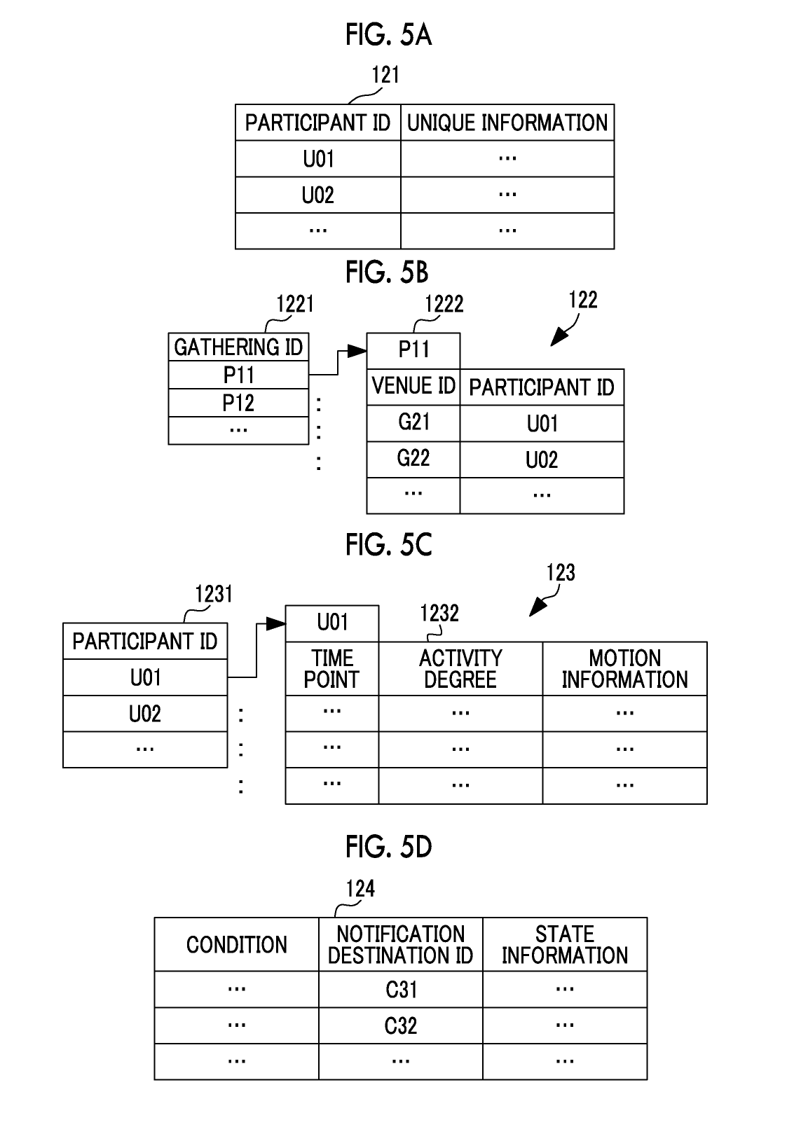

FIGS. 5A to 5D are diagrams illustrating a database stored in the storage unit 12. The participant DB 121 is a database in which information of a participant participating in a gathering is stored. The participant DB 121 illustrated in FIG. 5A stores a participant ID as identification information for identifying a participant, and unique information as information unique to the participant, in association with each other.

The unique information associated with the participant is, for example, information used for normalizing the activity degree or the motion amount of the participant or information for recognizing the participant. “Normalizing” the activity degree or the motion amount, which is described here, means processing of expressing the activity degree or the motion amount with, for example, a ratio using the maximum value, the minimum value, or the like of the activity degree or the motion amount which has been previously specified for the participant. Thus, an individual difference between the numerical values is canceled out. The information for recognizing the participant means that the information is presented by the terminal 2 and thereby presence of the participant is recognized. For example, such information refers to the name, the portrait, the icon, and the like of the participant.

The gathering DB 122 is a database in which information on a participant participating in a gathering is stored. The gathering DB 122 illustrated in FIG. 5B includes a gathering list 1221 and a venue list 1222.

The gathering list 1221 is a list of a gathering ID which is identification information of a gathering. In the venue list 1222, a participant ID indicating a participant in a gathering and a venue ID as identification information of a venue in which the participant actually moves are stored in association with each gathering ID.

The history DB 123 is a database in which information indicating how the activity degree and the motion information of a participant in a gathering have changed is stored. The history DB 123 illustrated in FIG. 5C includes a participant list 1231 and a history list 1232. The participant list 1231 is a list of a participant ID.

The history list 1232 is a list in which changes of the activity degree and the motion information of a participant identified by the corresponding participant ID, with time, are stored for each participant ID described in the participant list 1231. In the history list 1232, the activity degree and the motion information of a participant, which has been measured, observed, or specified at a time point are stored in association with each time point. That is, in the history list 1232 illustrated in FIG. 5C, history of a set of the activity degree specified for a participant and motion information of the participant when the activity degree has been specified is stored.

The state DB 124 is a database in which condition relating to the activity degree or the motion information, information (state information) indicating the state of a gathering when the condition is satisfied, and a notification destination ID are stored in associated with each other. The notification destination ID is identification information of a notification destination to be notified of the state information. The number of notification destinations identified by the notification destination ID may be one or plural. As a result of the condition, for example, in a case where negative state information that the activity degree or the motion amount deviates from the aim of the gathering, notification may be performed in a form different from that in a case where notification of positive state information is performed. The form of the notification means, for example, that only the chairman, not all participants, is notified.

Functional Configuration of Server Device

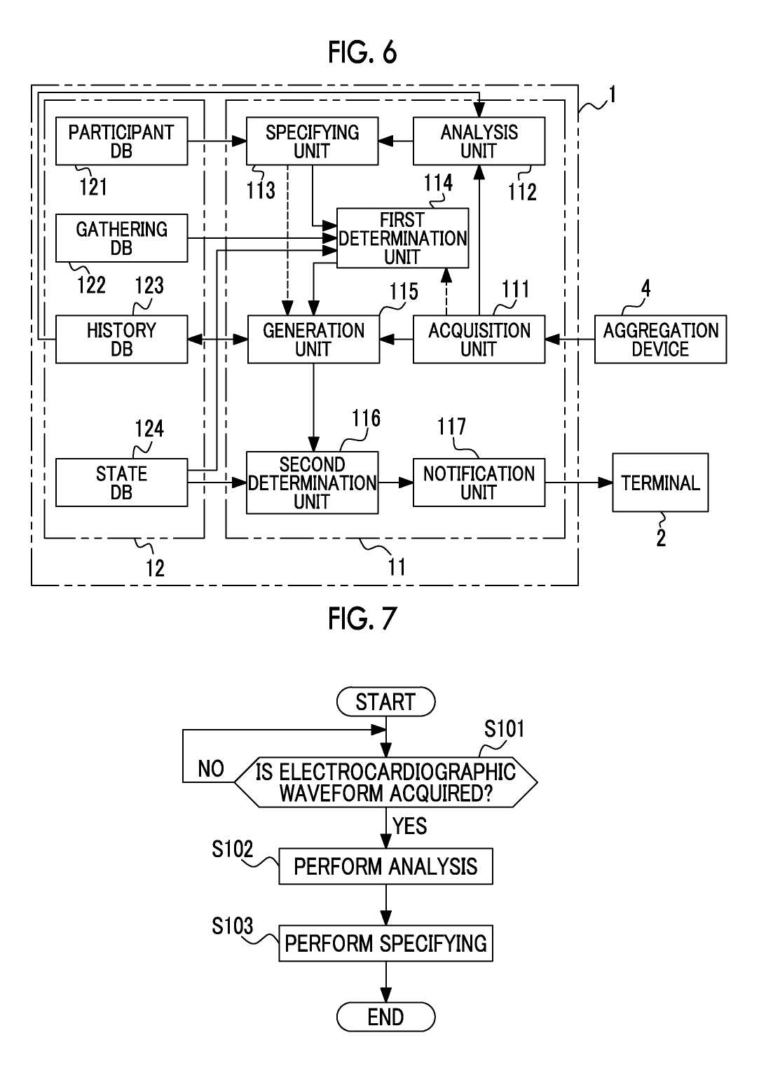

FIG. 6 is a diagram illustrating a functional configuration of the server device 1. The controller 11 of the server device 1 functions as an acquisition unit 111, an analysis unit 112, a specifying unit 113, a first determination unit 114, a generation unit 115, a second determination unit 116, and a notification unit 117, by reading and executing a program stored in the storage unit 12. In FIG. 6, illustrations of the communication line 3 and the communication unit 13 are omitted.

The acquisition unit 111 acquires biometric information of a participant U and motion information of the participant U from the aggregation device 4. Here, the biometric information indicates an electrocardiographic waveform which has been measured by electrodes of the measurement device 5 mounted on the participant U and has been transmitted to the server device 1 by the aggregation device 4. The motion information of the participant U is information which has been observed by the observation device 6 and has been transmitted to the server device 1 by the aggregation device 4. The motion information includes the motion amount of the participant.

The analysis unit 112 performs analysis for specifying the activity degree of the participant U based on the acquired biometric information. For example, the analysis unit 112 performs frequency analysis of the electrocardiographic waveform as the acquired biometric information so as to obtain frequency components of heart rate fluctuation, such as a very low frequency (VLF) component, a low frequency (LF) component, and a high frequency (HF) component.

Fast Fourier transform, Fourier series expansion with uneven intervals, wavelet transform, and the like are used in the analysis performed by the analysis unit 112. The analysis unit 112 may convert the time series of the electrocardiographic waveform by using, for example, cubic spline interpolation such that the R-R intervals are equally spaced. In a case where the acquisition unit 111 acquires biometric information (for example, a pulse wave and a heartbeat) of the participant other than the electrocardiographic waveform, the analysis unit 112 may analyze the biometric information.

LF is a word indicating a low frequency. A variable wave (blood pressure fluctuation) which is called as the Mayer wave and uses a blood pressure change having a period of about 10 seconds, as a signal source, or the total amount (integrated value) of the power spectrum in the frequency area is indicated by LF. The frequency area of LF is, for example, equal to or greater than 0.05 Hz and smaller than 0.15 Hz.

HF is a word indicating a high frequency. A variable wave (respiratory fluctuation) which uses respiration having a period of about 3 seconds to 4 seconds, as a signal source, or the total amount (integrated value) of the power spectrum in the frequency area is indicated by HF. The frequency area of HF is, for example, equal to or greater than 0.15 Hz and smaller than 0.40 Hz.

The autonomic nerves are classified into sympathetic nerves and parasympathetic nerves. In a case where the sympathetic nerves are excited, noradrenalin is released. The β receptor of a cell in the sinoatrial node receives the noradrenaline, and thereby a series of chemical reactions starts in the cell and the heart rate increases.

In a case where the parasympathetic nerves are excited, acetylcholine is released. The muscarinic receptor of the cell of the sinoatrial node receives the acetylcholine, and thereby a chemical reaction starts in the cell and the heart rate falls.

All of the chemical reactions of the sympathetic nerves and parasympathetic nerves have a mechanism of a reuse loop of chemical substances. Materials used in the reactions are recycled and return to the original states. The reaction time until the materials return to the original states is different between the sympathetic nerves and the parasympathetic nerves. Thus, the response speed for the transmitted signal is different.

For example, the chemical reaction of the sympathetic nerves, which involves noradrenalin and the β receptor has difficulty in responding to a change having a period which is shorter than a period of about 6 seconds. Therefore, in the sympathetic nerves, transmitting respiratory fluctuation having a period of about 3 seconds to 4 seconds is difficult.

The chemical reaction of the parasympathetic nerves, which involves acetylcholine and the muscarinic receptor proceeds faster than the chemical reaction of the sympathetic nerves. Thus, response to fluctuation having a period of about 1 second is also possible. Accordingly, the parasympathetic nerve transmits the respiratory fluctuation.

The blood pressure fluctuation has a period of about 10 seconds. Thus, all of the parasympathetic nerves and the sympathetic nerves may transmit the blood pressure fluctuation.

When the sympathetic nerve is suppressed, and the parasympathetic nerve is accelerated (tense), both the blood pressure fluctuation and the respiratory fluctuation are reflected in the heart rate fluctuation by the parasympathetic nerve. On the contrary, when the sympathetic nerve is accelerated, and the parasympathetic nerve is suppressed, the blood pressure fluctuation is reflected in the heart rate fluctuation by the sympathetic nerve, but the respiratory fluctuation is not reflected.

For the above reasons, the HF component reflecting the respiratory fluctuation appears in the heart rate fluctuation only in a case where the parasympathetic nerve is activated (excited). The LF component reflecting the blood pressure fluctuation also appears in the heart rate fluctuation in a case where any of the sympathetic nerve and the parasympathetic nerve is activated. Therefore, when the HF component is relatively large, it is understood that the parasympathetic nerve tends to be tense. When the LF component is relatively large, it is understood that the sympathetic nerve tends to be tense. For example, a value (LF/HF) obtained by dividing the LF component by the HF component represents the activity degree of the sympathetic nerve and is handled as an index of the strength of stress.

The specifying unit 113 specifies the activity degree of the participant U based on results of the analysis performed by the analysis unit 112. That is, the specifying unit 113 specifies the activity degree of a participant, which is analyzed based on biometric information obtained from the living body of the participant participating in a gathering.

The activity degree of the participant U is an index indicating how active the participant U acts. The activity degree of the participant U indicates the concentration degree of the participant U, the depth of understanding, and the like. The activity degree is derived as a function of a value (for example, LF component and HF component which are described above) which is obtained by the specifying unit 113 from the frequency analysis of an electrocardiographic waveform, which has been performed by the analysis unit 112. Examples of the activity degree include the sum of the LF component and the HF component. The activity degree may be normalized for each participant with the specifying unit 113 referring to the participant DB 121.

In the following descriptions, a numerical value obtained by normalization with the minimum value of 0 and the maximum value of 1 is used as the activity degree.

The first determination unit 114 specifies plural participants participating in a gathering as a target of determination, with reference to the gathering DB 122. The first determination unit 114 determines whether or not activity degrees of the specified participants satisfy a condition (first condition) determined at a predetermined percentage, with reference to the state DB 124.

The predetermined percentage is, for example, 70% (percentage) of all participants. The first condition is a condition that the activity degree is equal to or greater than a threshold of, for example, 0.6. That is, the meaning that the activity degrees of plural participants satisfy the first condition at the predetermined percentage is that, for example, the specified activity degrees of 70% of the participants are equal to or greater than 0.6.

The generation unit 115 generates information (correlation information) indicating a correlation between the motion information and the activity degree of the participant. The generation unit 115 illustrated in FIG. 6 generates correlation information including a correlation coefficient by calculating the correlation coefficient between the motion information and the activity degree, for each of participants determined to satisfy the first condition at the above-described percentage by the first determination unit 114. That is, the generation unit 115 generates correlation information of a participant when the activity degree of the participant satisfies the first condition.

Here, even in a case where the correlation coefficient is equal to or greater than a threshold and it is recognized that the motion information and the activity degree has a correlation, determining a causal relationship between the motion information and the activity degree is not possible. The reason is that cases as follows are considered: a case where the motion information and the activity degree accidentally show the common tendency; and a case where the motion information and the activity degree show the common tendency due to a common cause. A case in which the direction of causality is reversed even though the correlation relationship is recognized, and the causal relationship is recognized is also considered. However, in a case where the correlation is recognized, for example, there is a probability of the causal relationship (that is, the activity degree of a participant increases by an operation of the participant himself/herself) being provided.

The generation unit 115 may also generate correlation information between the motion information and the activity degree for a participant other than the participants having an activity degree determined to satisfy the first condition at the above-described percentage. Since the server device generates correlation information for the participants having an activity degree determined to satisfy the first condition at the above-described percentage, a calculation load is reduced in comparison to a case of generating correlation information for all the participants.

The second determination unit 116 determines whether or not pieces of correlation information of participants satisfy the predetermined second condition when the activity degrees of the participants satisfy the first condition at the above-described percentage, with reference to the state DB 124. The second condition is a condition, for example, that the correlation coefficient between the motion amount and the activity degree of the participant is smaller than a threshold of 0.6 or the like. The second condition may be a condition relating to the correlation coefficient (calculated for one participant) between the motion amount and the activity degree or may be a condition relating to the correlation coefficient for plural participants.

The notification unit 117 notifies a notification destination which is indicated by a notification destination ID associated with a condition, of state information associated with the condition in a case where the condition stored in the state DB 124 is satisfied. The state information is information indicating the state of the gathering when the condition is satisfied. The state information includes information corresponding to the determination result of the second determination unit 116. The state information may include unique information for recognizing one person or plural participants in a case where the condition is satisfied.

Operation of Server Device

FIG. 7 is a flowchart illustrating a flow of an operation in which the server device 1 specifies the activity degree. The controller 11 of the server device 1 determines whether or not an electrocardiographic waveform (example of biometric information) of a participant is acquired from the aggregation device 4 (Step S101). The controller 11 continues this determination during a period (NO in Step S101) of determining that the electrocardiographic waveform is not acquired. In a case where it is determined that the electrocardiographic waveform of the participant has been acquired (YES in Step S101), the controller 11 analyzes the electrocardiographic waveform (Step S102). The controller 11 specifies the activity degree of the participant based on an analysis result obtained by analyzing the electrocardiographic waveform (Step S103).

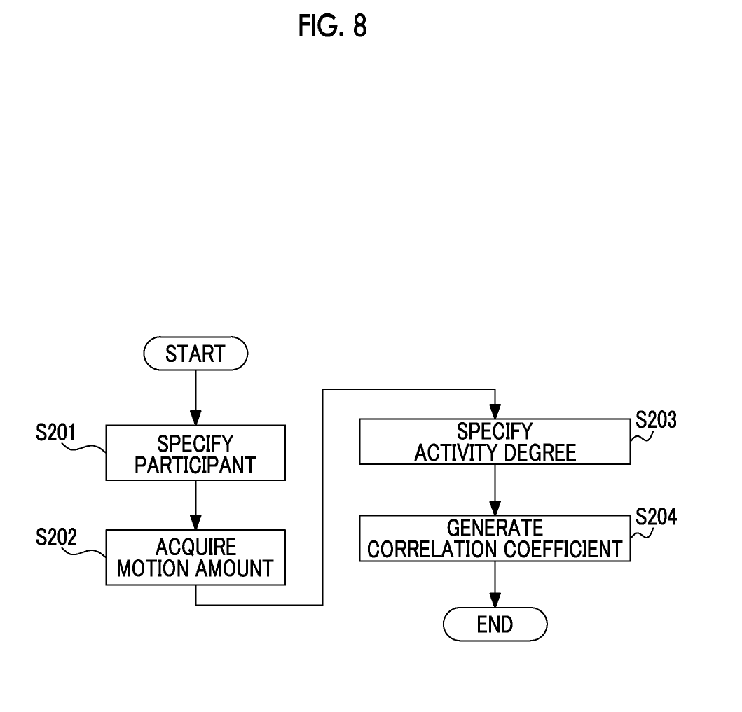

FIG. 8 is a flowchart illustrating a flow of an operation of the server device 1 generating a correlation coefficient. The controller 11 of the server device 1 specifies a participant in the gathering with reference to the gathering DB 122 (Step S201). The controller 11 acquires the motion amount acquired for each specified participant (Step S202) and specifies the activity degree of the participant at this time (Step S203). The order of Step S202 and Step S203 may be reversed. The controller 11 generates a correlation coefficient as correlation information indicating a correlation between the acquired motion amount and the specified activity degree (Step S204).

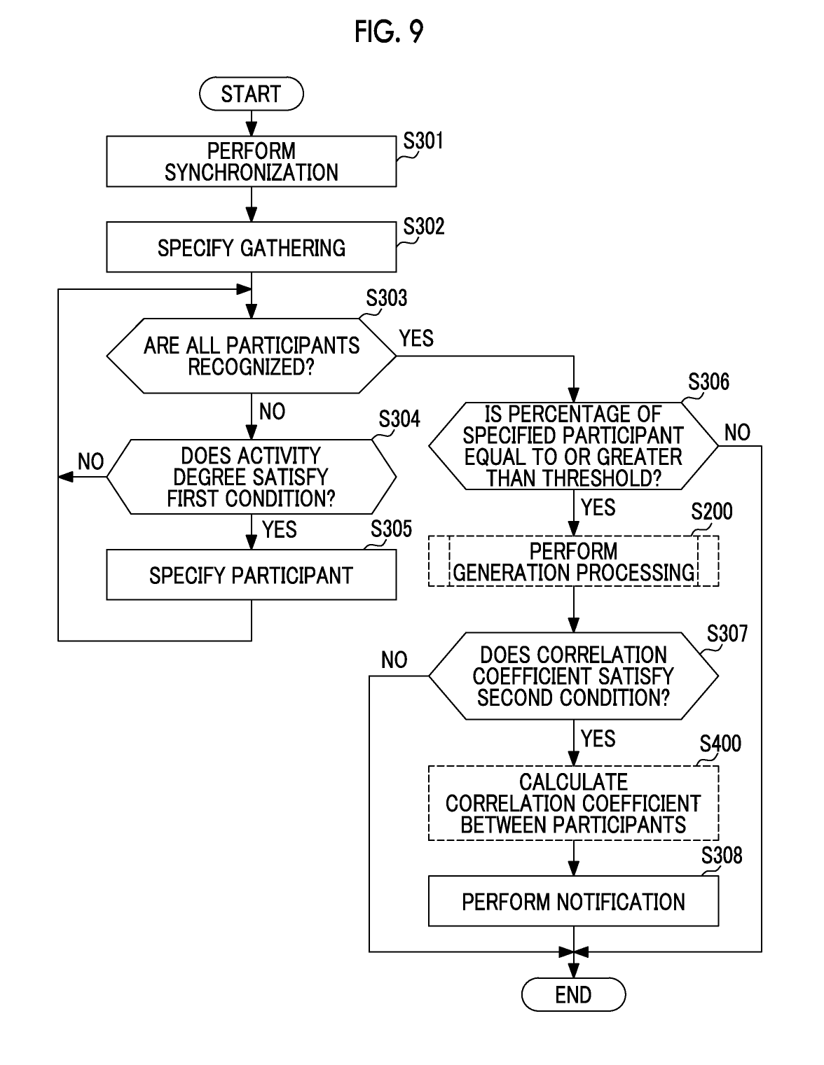

FIG. 9 is a flowchart illustrating a flow of an operation of the server device 1 performing notification of state information of a gathering. The controller 11 of the server device 1 synchronizes the motion information acquired for each participant with the specified activity degree (Step S301). The controller 11 specifies the gathering as a target by sequentially selecting the gathering registered in the gathering DB 122, for example (Step S302).

The controller 11 determines whether or not recognizing all participants of the specified gathering is completed (Step S303). In a case where it is determined that the recognizing is not completed (NO in Step S303), the controller 11 determines whether or not the activity degree of the participant who is not recognized satisfies the first condition (Step S304).

In a case where it is determined the activity degree of the participant satisfies the first condition (YES in Step S304), the controller 11 specifies the participant (Step S305). In a case where it is determined that the activity degree of the participant does not satisfy the first condition (NO in Step S304), the controller 11 causes the process to return to Step S303.

In a case where it is determined that the recognizing is completed (YES in Step S303), the controller 11 determines whether or not the percentage of the participants specified in Step S305 is equal to or greater than a threshold (Step S306). In a case where it is determined that the percentage of the participants is not equal to or greater than the threshold (NO in Step S306), the controller 11 ends the process.

In a case where it is determined that the percentage of the participants is equal to or greater than the threshold (YES in Step S306), the controller 11 performs generation processing illustrated in FIG. 8, for the specified participant (Step S200). The controller 11 determines whether or not the generated correlation coefficient satisfies the second condition (Step S307).

The controller 11 may also generate the correlation coefficient between the motion amount and the activity degree of a participant other than the specified participant.

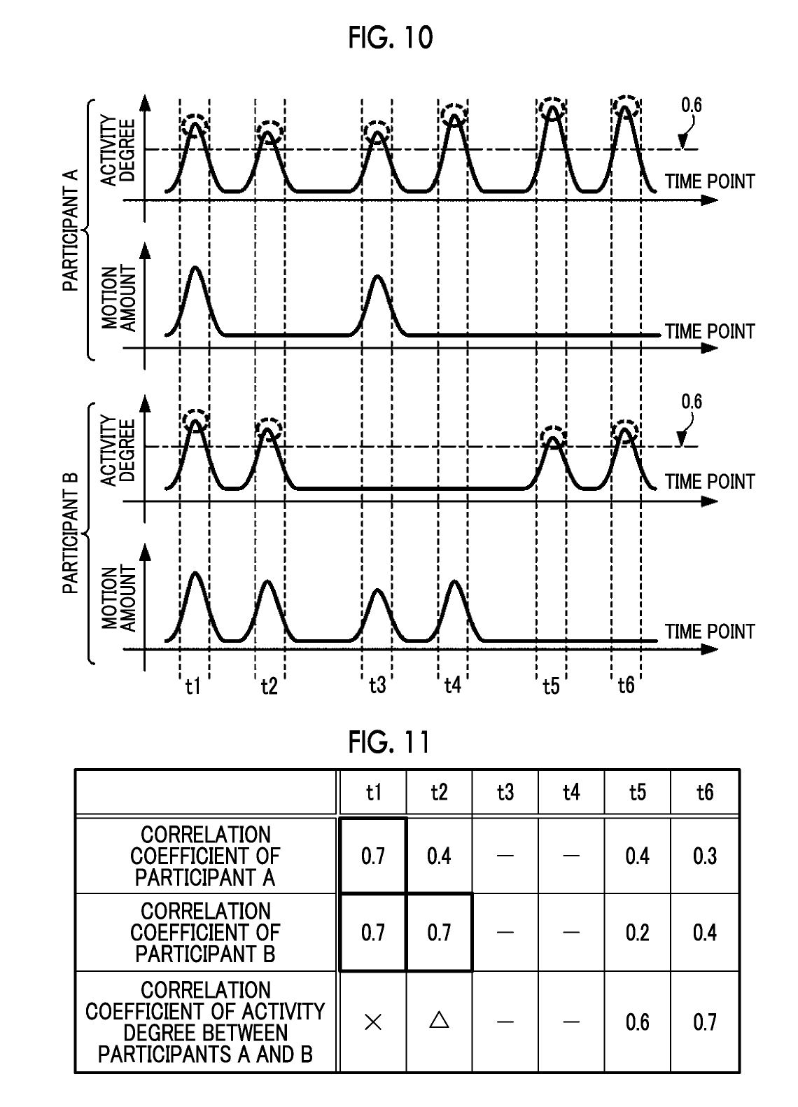

FIG. 10 is a diagram illustrating an example of changes of the activity degree and the motion amount of a participant with time. In FIG. 10, a horizontal axis indicates a time and a vertical axis indicates the activity degree or the motion amount. In this example, two participants of a participant A and a participant B participate in the gathering.

For example, the controller 11 determines whether or not the activity degree normalized for each participant is equal to or greater than 0.6 as the threshold, as the first condition. The activity degree of the participant A is equal to or greater than 0.6 as the threshold in each of periods t1, t2, t3, t4, t5, and t6. Thus, it is determined that the first condition is satisfied in each of the periods. The activity degree of the participant B is equal to or greater than 0.6 in each of the periods t1, t2, t5, and t6, but is smaller than 0.6 in the periods t3 and t4.

Here, in a case where the above-described percentage is set to 100%, the controller 11 determines that the percentage of the participants having an activity degree satisfying the first condition is equal to or greater than the threshold in the periods t1, t2, t5, and t6. That is, in a case where the operation illustrated in FIG. 9 is performed on the gathering illustrated in FIG. 10, the controller 11 specifies the participant A and the participant B in the periods t1, t2, t5, and t6. The controller 11 generates the correlation coefficient of each of the participant A and the participant B in these periods and determines whether or not the generated correlation coefficient satisfies the second condition.

In a case where it is determined that the generated correlation coefficient does not satisfy the second condition (NO in Step S307), the controller 11 ends the process. In a case where it is determined that the generated correlation coefficient satisfies the second condition (YES in Step S307), the controller 11 calculates the correlation coefficient between the participants (Step S400).

At this time, the controller 11 selects two of the participants in the gathering and calculates the correlation coefficient of the activity degree between the selected participants. Regarding selection of the participants, for example, a participant having an activity degree showing the minimum value and a participant having an activity degree showing the maximum value are selected among the participants of the gathering.

When participants of the gathering satisfy the first condition at the predetermined percentage and any of the participants satisfies the second condition, the controller 11 notifies the terminal 2 as the associated notification destination, of state information of the gathering, which is associated with satisfaction of the conditions (Step S308). Then, the controller 11 ends the process. In this case, the state information of which notification is performed may include a correlation coefficient of the activity degree between two participants selected from plural participants, when the correlation information satisfies the second condition.

In a case where notification of only a message indicating that each of the above-described conditions is satisfied, as the state information of the gathering, is performed, the controller 11 may not calculate the correlation coefficient between the participants. The controller 11 may calculate a statistical amount such as kurtosis indicating the sharpness of frequency distribution of the activity degree of the participant in the gathering, instead of or in addition to calculation of the correlation coefficient between the participants. For example, kurtosis for the motion amount of each of participants may be calculated as an index indicating the correlation between the participants. In short, the controller 11 may calculate an index indicating the common tendency of the activity degree or the motion amount between the participants.

FIG. 11 is a diagram illustrating the state information of which notification is performed. When the activity degree and the motion amount of the participant in the gathering change with time as illustrated in FIG. 10, as described above, the controller 11 specifies the participant A and the participant B in the periods t1, t2, t5, and t6 and calculates the correlation coefficient between the activity degree and the motion amount for each of the participants.

Regarding the correlation coefficient between the activity degree and the motion amount in the period t1, the participant A is 0.7 and the participant B is also 0.7. This means that no one satisfies the second condition of “being smaller than 0.6”. At this time, the controller 11 may notify the terminal 2 as the predetermined notification destination, of a possibility that the activity degree seems to have increased only apparently because the participant A and the participant B together perform a predetermined motion such as standing-up, in the period t1. At this time, the controller 11 may not perform the notification.

Regarding the correlation coefficient between the activity degree and the motion amount in the period t2, the participant A is 0.4 and the participant B is 0.7. In this case, the activity degree of the participant A satisfies the second condition and the activity degree of the participant B does not satisfy the second condition. At this time, the controller 11 may notify the terminal 2 as the predetermined notification destination of a message as follows: a message indicating that, in the period t2, regarding the participant A, a possibility that the activity degree seems to increase at least by an influence of the motion because the activity degree having a low correlation with the motion amount is equal to or greater than the threshold is low; or a message indicating that, in the period t2, regarding the participant B, a possibility that the activity degree seems to increase by the influence of the motion because the activity degree having a high correlation with the motion amount is equal to or greater than the threshold is high. At this time, the controller 11 may not perform the notification.

Regarding the correlation coefficient between the activity degree and the motion amount in the period t5, the participant A is 0.4 and the participant B is 0.2. This means that all the participants satisfy the second condition of “being smaller than 0.6”. At this time, the controller 11 calculates the correlation coefficient of the activity degree between the participant A and the participant B and notifies the terminal 2 of the calculated correlation coefficient. The correlation coefficient between the participants illustrated in FIG. 11 is 0.6 and this value is transmitted to a terminal 2 of, for example, the chairman of the gathering, as an index of mutual understanding.

Regarding the correlation coefficient between the activity degree and the motion amount in the period t6, the participant A is 0.3 and the participant B is 0.4. Thus, all the participants satisfy the second condition. At this time, the controller 11 also calculates the correlation coefficient of the activity degree between the participant A and the participant B and notifies the terminal 2 of the calculated correlation coefficient. As illustrated in FIG. 11, in the period t6, the correlation coefficient between the participants is 0.7 and this value is transmitted to the terminal 2 of, for example, the chairman of the gathering, as the index of mutual understanding.

With the above operation, even in a case where the activity degree of the participant in the gathering seems to increase by the motion of the participant, the server device 1 distinguishes this case from a case where the activity degree increases when an influence of the motion is not applied. Thus, an accuracy at which the user of the information processing system 9 determines whether or not the participant in the gathering really concentrates on the gathering is improved. The user of the information processing system 9 grasps the period in which participants of the predetermined percentage or greater among participants in the gathering concentrate on the gathering, regardless of the small motion amount.

In a case where the server device 1 is notified of the current state of the gathering in real time, the host of the gathering such as the chairman or a facilitator, who receives the notification progresses the gathering with reference to the contents of which notification has been performed. As a result, for example, even in a gathering in which it seems that mutual understanding apparently proceeds, the host waits for a statement without proceeding the agenda in advance or urges a participant who is specified to have a low correlation between the motion information and the activity degree to make statement. Thus, it is easy to achieve the purpose of the gathering.

The server device 1 may be used for analyzing a gathering which has been previously performed, instead of receiving the notification in real time. The analysis result may be reproduced in synchronization with a video or audio of a participant which has participated in the previous gathering, for example.

### MODIFICATION EXAMPLES

Although the above descriptions are made for the exemplary embodiment, details of the exemplary embodiment may be modified as follows. The following modification examples may be combined.

### Modification Example 1

In the above-described exemplary embodiment, the second determination unit 116 determines whether or not the correlation information of the participant satisfies the predetermined second condition. However, as the second condition, plural conditions may be provided. That is, the second determination unit 116 may determine whether or not the correlation information of the participant satisfies each of the plural second conditions.

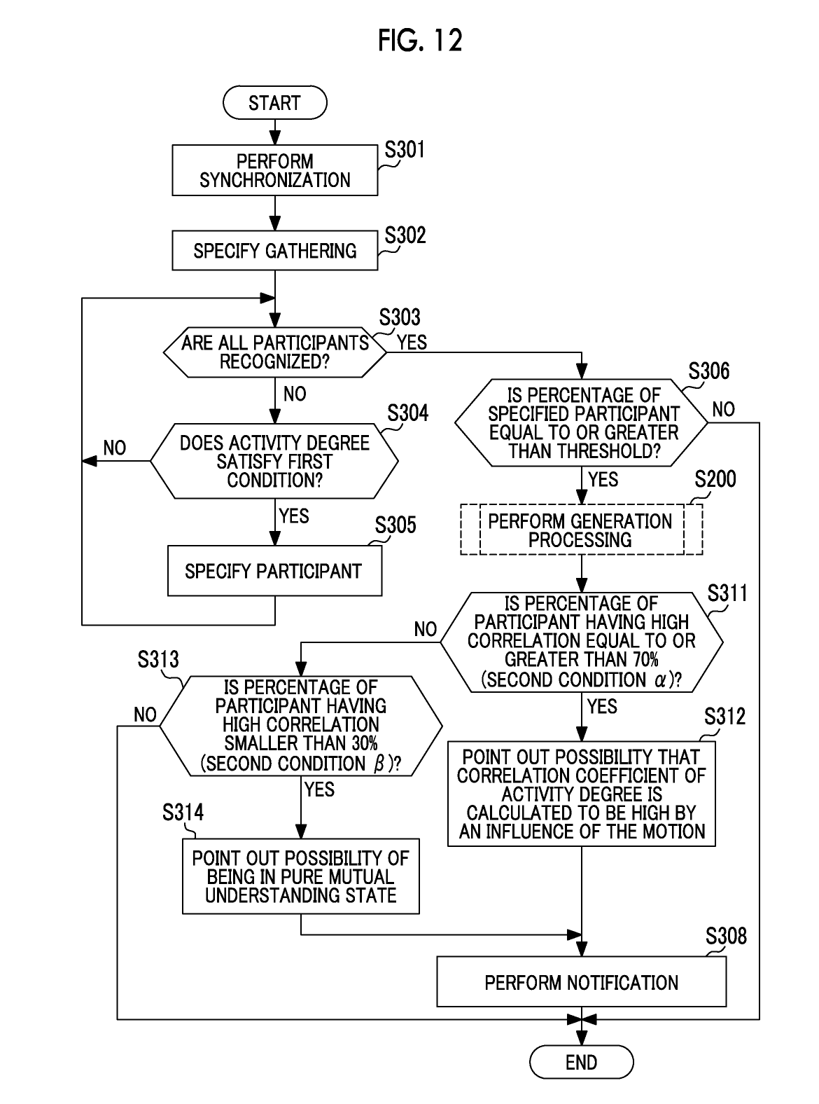

FIG. 12 is a flowchart illustrating a flow of an operation of the server device 1 performing notification of state information of the gathering in a modification example. Descriptions of steps which are illustrated in FIG. 12 and are common with the steps in FIG. 9 will not be repeated.

In Step S306, in a case where it is determined that the percentage of the participants is equal to or greater than the threshold (YES in Step S306), the controller 11 performs generation processing illustrated in FIG. 8, for the specified participant (Step S200). The controller 11 determines whether or not the generated correlation coefficient satisfies the second condition (Step S311).

In the modification example, the second condition is divided into two. Here, a second condition used in determination in Step S311 is set as a second condition α and another second condition is set as a second condition 13.

The second condition α illustrated in FIG. 12 is a condition in which the percentage of participants having a correlation coefficient between the motion amount and the activity degree, which is equal to or greater than the threshold (also referred to as “a participant having a high correlation” below), to all participants of the gathering is equal to or greater than the predetermined percentage of, for example, 70%.

In a case where it is determined that the percentage of the participants having a high correlation is equal to or greater than 70% (YES in Step S311), the controller 11 includes, for example, words that it is pointed out that there is a possibility of the correlation coefficient of the activity degree being calculated to be high by an influence of the motion, in state information (Step S312).

In a case where it is determined that the percentage of the participants having a high correlation is not equal to or greater than 70% (NO in Step S311), the controller 11 determines whether or not the correlation coefficient satisfies the second condition β (Step S313). The second condition β illustrated in FIG. 12 is a condition in which the percentage of the participants having a high correlation to all of the participants in the gathering is smaller than the predetermined percentage of, for example, 30%.

In a case where it is determined that the percentage of the participants having a high correlation is smaller than 30% (YES in Step S313), the controller 11 includes, for example, words that it is pointed out that there is a possibility of the state of the gathering being a simple mutual understanding state, in the state information (Step S314).

The controller 11 notifies the terminal 2 as the predetermined notification destination, of the state information generated in Step S312 or Step S314 (Step S308).

In a case where it is determined that the percentage of the participants having a high correlation is not smaller than 30% (NO in Step S313), the controller 11 ends the process without performing notification in Step S308.

With the above operation, the server device 1 determines whether or not the correlation information of the participant satisfies each of the plural second conditions, and thus the user is notified of the state information corresponding to the determination result. Therefore, the user gets more detailed information on the state of the gathering.

### Modification Example 2

In the exemplary embodiment or the modification example which is described above, the second condition is predetermined. However, the second condition may be determined in accordance with a statistical value calculated based on the previous correlation information of the participant. Examples of the statistical value include the maximum value, the minimum value, the median value, the average value, variance, and standard deviation.

### Modification Example 3

The second condition may be determined for each participant, in accordance with the statistical value calculated based on the previous correlation information of each participant. In this case, the threshold compared to the correlation coefficient or the like in the second condition is determined for each participant. Thus, an individual difference of the correlation coefficient between the activity degree and the motion amount may be canceled out.

### Modification Example 4

In the above-described exemplary embodiment, the generation unit 115 generates the correlation information between the motion information and the activity degree. However, the generation unit 115 may generate correlation information indicating a correlation between the activity degree and environmental information indicating the surrounding environment of the participant, instead of or in addition to the motion information. For example, the environmental information means information obtained from the surrounding environment of the participant, such as the temperature or humidity of a venue in which the participant is located, a wind direction or an air volume, smell, and vibration. In this case, the observation device 6 may observe the above-described environmental information.

Events indicating the environmental information and the motion information are not observation events (psychogenic events) caused by the psychological state of the participant but are observation events (such as an environment or a motion) caused by a state other than the psychological state. Thus, the above events are also defined as non-psychogenic events.

### Modification Example 5

In the above-described exemplary embodiment, the controller 11 determines whether or not recognizing all participants in the specified gathering is completed. In a case where it is determined that the recognizing is not completed, the controller 11 determines whether or not the activity degree of the participant who is not recognized satisfies the first condition. However, the controller 11 may determine whether or not the motion information of the participant satisfies a predetermined condition (third condition).

The third condition may be determined, for example, for the motion amount of the participant in the motion information. In this case, the motion amount may be normalized for each participant with the specifying unit 113 referring to the participant DB 121. In the following descriptions, a numerical value obtained by normalization with the minimum value of 0 and the maximum value of 1 is used as the motion amount.

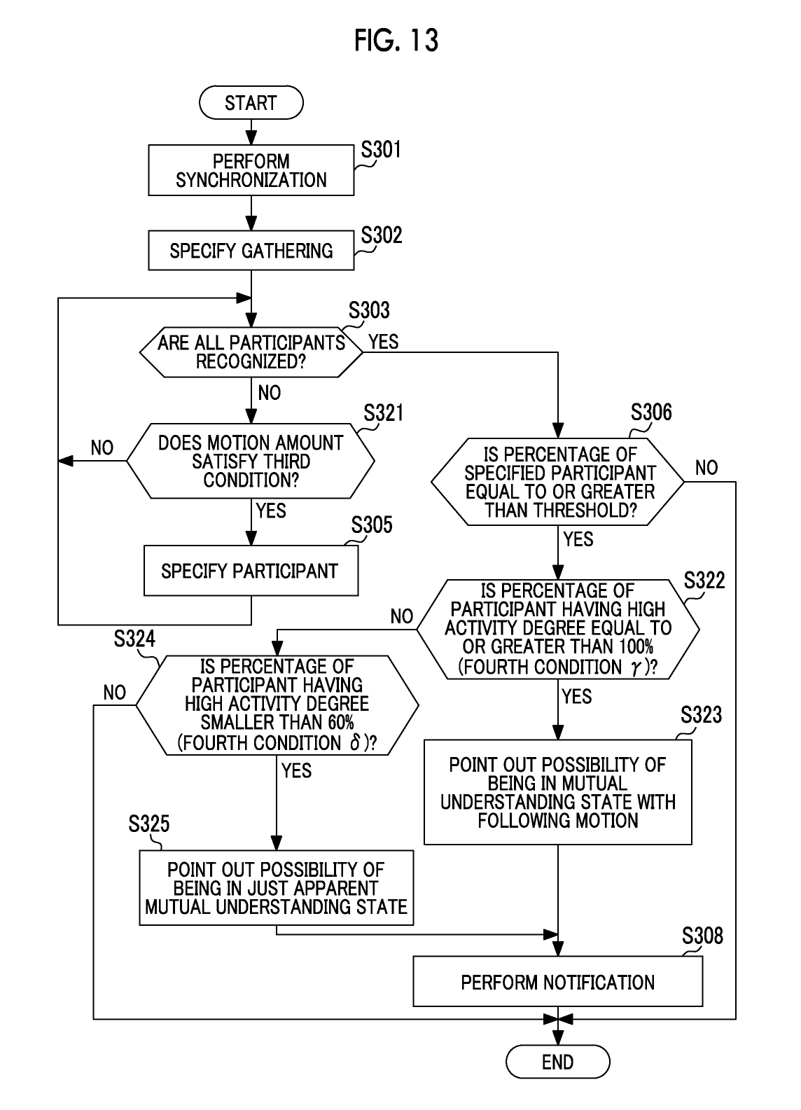

FIG. 13 is another flowchart illustrating a flow of an operation of the server device 1 performing notification of state information of the gathering in a modification example. Descriptions of steps which are illustrated in FIG. 13 and are common with the steps in FIG. 9 will not be repeated.

The controller 11 determines whether or not recognizing all participants of the specified gathering is completed (Step S303). In a case where it is determined that the recognizing is not completed (NO in Step S303), the controller 11 determines whether or not the motion amount of the participant who is not recognized satisfies the third condition (Step S321). For example, as the third condition, whether or not the motion amount is equal to or greater than 0.6 may be provided.

In this case, the first determination unit 114 which is realized in the controller 11 by executing the program determines whether or not pieces of motion information of plural participants participating in the gathering satisfy the predetermined third condition.

In a case where it is determined the motion amount of the participant satisfies the third condition (YES in Step S304), the controller 11 specifies the participant (Step S305). In a case where it is determined that the motion amount of the participant does not satisfy the third condition (NO in Step S304), the controller 11 causes the process to return to Step S303.

In Step S306, in a case where it is determined that the percentage of the participants is equal to or greater than the threshold (YES in Step S306), the controller 11 determines whether or not the generated correlation coefficient satisfies a fourth condition (Step S322).

In this case, the second determination unit 116 which is realized in the controller 11 by executing the program determines whether or not the activity degree of the participant satisfies the predetermined fourth condition, when the motion information satisfies the third condition.

In the modification example, the fourth condition is divided into two. Here, a fourth condition used in determination in Step S322 is set as a fourth condition γ and another fourth condition is set as a fourth condition δ.

The fourth condition γ illustrated in FIG. 13 is a condition in which the percentage of participants having an activity degree which is equal to or greater than the threshold (also referred to as “a participant having a high activity degree” below) to all participants of the gathering is equal to or greater than the predetermined percentage of, for example, 100%.

In a case where it is determined that the percentage of the participants having a high activity degree is equal to or greater than 100% (YES in Step S322), the controller 11 includes, for example, words that it is pointed out that there is a possibility of the state of the gathering being a pure mutual understanding state with following the motion, in the state information (Step S323).

In a case where it is determined that the percentage of the participants having a high activity degree is not equal to or greater than 100% (NO in Step S322), the controller 11 determines whether or not the correlation coefficient satisfies the fourth condition δ (Step S324). The fourth condition δ illustrated in FIG. 12 is a condition in which the percentage of the participants having a high activity degree to all of the participants in the gathering is smaller than the predetermined percentage of, for example, 60%.

In a case where it is determined that the percentage of the participants having a high activity degree is smaller than 60% (YES in Step S324), the controller 11 includes, for example, words that it is pointed out that there is a possibility of the state of the gathering being just an apparent mutual understanding state, in the state information (Step S325).

The controller 11 notifies the terminal 2 as the predetermined notification destination, of the state information generated in Step S323 or Step S325 (Step S308).

That is, the notification unit 117 which is realized in the controller 11 by executing the program performs notification of the state information indicating the state of the gathering, by using the determination result of the second determination unit 116.

In a case where it is determined that the percentage of the participants having a high activity degree is not smaller than 60% (NO in Step S324), the controller 11 ends the process without performing notification in Step S308.

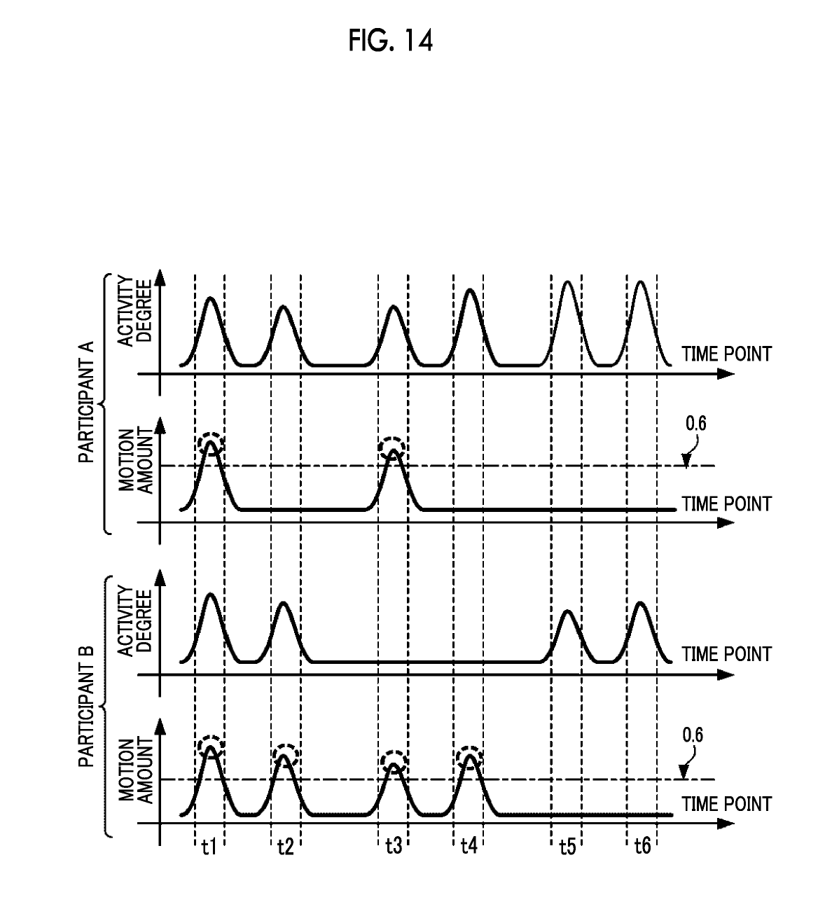

FIG. 14 is a diagram illustrating an example of changes of the activity degree and the motion amount of the participant with time, in the modification example. In FIG. 14, a horizontal axis indicates a time and a vertical axis indicates the activity degree or the motion amount. In this example, two participants of a participant A and a participant B participate in the gathering.

For example, the controller 11 determines whether or not the motion amount normalized for each participant is equal to or greater than 0.6 as the threshold, as the third condition. The motion amount of the participant A is equal to or greater than 0.6 as the threshold in each of the periods t1 and t3. Thus, it is determined that the third condition is satisfied in each of the periods. The motion amount of the participant B is equal to or greater than 0.6 in each of the periods t1, t2, t3, and t4. Thus, it is determined that the third condition is satisfied in each of the periods.

Here, in a case where the above-described percentage is set to 100%, the controller 11 determines that the percentage of the participants having a motion amount satisfying the third condition is equal to or greater than the threshold in the periods t1 and t3. That is, in a case where the operation illustrated in FIG. 13 is performed on the gathering illustrated in FIG. 14, the controller 11 specifies the participant A and the participant B in the periods t1 and t3. The controller 11 determines whether or not the motion amount of each of the participant A and the participant B satisfies the fourth condition in these periods.

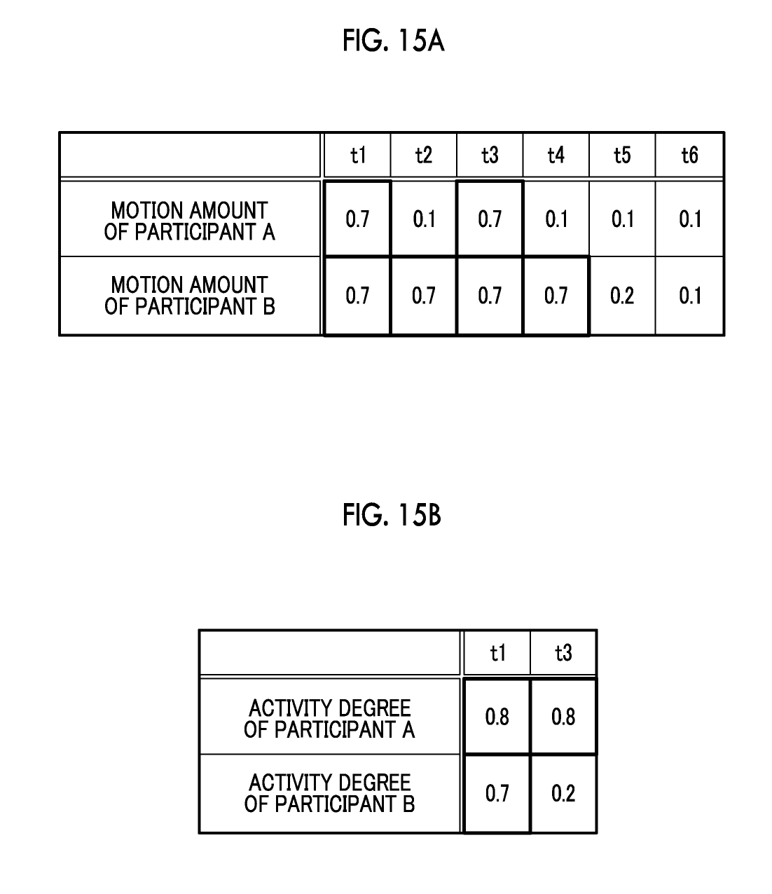

FIGS. 15A and 15B are diagrams illustrating the state information of which notification is performed. When the activity degree and the motion amount of the participant in the gathering change as illustrated in FIG. 14, the motion amounts of the participant A and the participant B are as illustrated in FIG. 15A. In this case, as described above, the controller 11 specifies the participant A and the participant B in the periods t1 and t3 and compares the activity degree of each of the participants to the threshold.

As illustrated in FIG. 15B, regarding the activity degree in the period t1, the participant A is 0.8 and the participant B is 0.7. This means the followings. That is, since the activity degrees of all of the participants are equal to or greater than the threshold of 0.6, all of the participant A and the participant B are participants having a high activity degree and the percentage of the participants having a high activity degree in the gathering is 100%.

At this time, the controller 11 notifies the terminal as the predetermined notification destination, of a possibility of being in a mutual understanding state with following the motion since the participant A and the participant B perform a motion at a predetermined level in the period t1, but an increase of the activity degree is also recognized.

Regarding the activity degree in the period t3, the participant A is 0.8 and the participant B is 0.2. In this case, since the activity degree of the participant A is equal to or greater than the threshold of 0.6, the participant A is a participant having a high activity degree. However, since the activity degree of the participant B is smaller than the threshold of 0.6, the participant B is not a participant having a high activity degree. That is, the percentage of the participants having a high activity degree in the gathering is 50% and is smaller than 60%.

At this time, the controller 11 notifies the terminal 2 as the predetermined notification destination of a message indicating that, regarding the participant Bin the period t3, there is a possibility of a state in which the motion is deviated from the mentality (for example, the participant appears to agree but does not have understanding) because the activity degree is smaller than the threshold even though the motion amount is equal to or greater than the threshold.

With the above operation, when motion amounts of participants of the above-described percentage or greater among the participants in the gathering are equal to or greater than the threshold (satisfy the third condition), the server device 1 determines whether or not the participant has a high activity degree, that is, determines whether or not the activity degree of the participant satisfies the fourth condition. Thus, the server device 1 determines whether or not the relatively high motion amount follows the increase of the activity degree. As a result, the user knows a case where the motion amount does not follow the increase of the activity degree although the motion amount increases, and the accuracy at which the user of the information processing system 9 determines the state of the gathering is improved.

### Modification Example 6

In the above-described modification example 5, the controller 11 may generate the correlation coefficient between the motion amount and the activity degree for each participant, for example. In this case, the controller 11 may determine whether or not the generated correlation coefficient satisfies the above-described fourth condition. The fourth condition means, for example, that the percentage of the participants who have a correlation coefficient of 0.6 or greater and has a high correlation is equal to or greater than the predetermined percentage or is smaller than the predetermined percentage.

That is, the generation unit 115 which is realized in the controller 11 by executing the program generates the correlation information indicating the correlation between the motion information and the activity degree. The second determination unit 116 realized in the controller 11 determines whether or not the generated correlation information satisfies the fourth condition.

### Modification Example 7

In the above-described exemplary embodiment, the controller 11 functions as the acquisition unit 111, the analysis unit 112, the specifying unit 113, the first determination unit 114, the generation unit 115, the second determination unit 116, and the notification unit 117, by reading and executing the program stored in the storage unit 12. However, any of the functions may be performed in the terminal 2 or the aggregation device 4.

For example, in a case where the controller 41 acquires biometric information from the measurement device 5, the controller 41 of the aggregation device 4 realizes the function corresponding to the above-described acquisition unit 111.

The controller 41 of the aggregation device 4 may perform analysis for specifying the activity degree of a participant based on the acquired biometric information. That is, the controller 41 may realize the function corresponding to the above-described analysis unit 112. In this case, the controller 41 may transmit information indicating a result obtained by the analysis to the server device 1. The controller 11 of the server device 1 may specify the activity degree of the participant based on the received information.

In the information processing system 9, the terminal 2 may perform the function of the aggregation device 4 together. In the above-described exemplary embodiment, although the communication line 3 links plural venues, the information processing system 9 may be applied to a gathering held in one venue. In this case, the server device 1 may perform the functions of the terminal 2 and the aggregation device 4 and the communication line 3 may not be provided.

### Modification Example 8

In the above-described exemplary embodiment, the notification unit 117 transmits the state information of the gathering to the terminal 2. However, in addition to or instead of the transmission of the state information, the notification unit 117 may transmit a control signal in a case of acquiring the information of the specified participant, to the aggregation device 4.

For example, the notification unit 117 may transmit a control signal for adjusting the focal length of an optical system that performs imaging of a participant, in accordance with the activity degree or the motion amount of the participant or the correlation coefficient between the activity degree and the motion amount, to the aggregation device 4. In this case, the aggregation device 4 transmits the transmitted control signal to the imaging device (not illustrated), and the imaging device performs the adjustment in accordance with the control signal.

The notification unit 117 may transmit a control signal for adjusting directivity to collect voice of a participant, in accordance with the activity degree or the motion amount of the participant or the correlation coefficient between the activity degree and the motion amount, to the aggregation device 4. In this case, the aggregation device 4 transmits the transmitted control signal to the sound collection device (not illustrated), and the sound collection device performs the adjustment in accordance with the control signal.

### Modification Example 9

The program executed by the controller 11 of the server device 1 may be provided in a state of being stored in a recording medium readable by a computer device, such as a magnetic recording medium (for example, a magnetic tape and a magnetic disk), an optical recording medium (for example, an optical disk), a magneto-optical recording medium, and a semiconductor memory. The program may be downloaded via a communication line such as the Internet. Various devices other than the CPU may be applied as a control section exemplified by the above-described controller 11. For example, a dedicated processor or the like is used.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing apparatus comprising:
a specifying unit that specifies an activity degree of a participant based on biometric information obtained from a living body of the participant participating in a gathering;
an acquisition unit that acquires motion information indicating a motion of the participant;
a generation unit that generates correlation information indicating a correlation between the motion information and the activity degree; and
a notification unit that performs a notification of state information indicating a state of the gathering by using the correlation information.

2. The information processing apparatus according to claim 1, further comprising:
a first determination unit that determines whether or not activity degrees of a plurality of participants participating in the gathering satisfy a predetermined first condition at a predetermined percentage; and
a second determination unit that determines whether or not the correlation information of the participant satisfy a predetermined second condition when the activity degrees satisfy the first condition at the predetermined percentage,
wherein the state information includes information corresponding to a determination result of the second determination unit.

3. The information processing apparatus according to claim 2,
wherein the generation unit generates the correlation information of each of the participants when the activity degree satisfies the first condition.

4. The information processing apparatus according to claim 2,
wherein the state information includes a correlation coefficient between the activity degrees of two participants selected from the plurality of participants when the correlation information satisfies the second condition.

5. The information processing apparatus according to claim 3,
wherein the state information includes a correlation coefficient between the activity degrees of two participants selected from the plurality of participants when the correlation information satisfies the second condition.

6. The information processing apparatus according to claim 2,
wherein the second determination unit determines whether or not the correlation information satisfies each of a plurality of second conditions.

7. The information processing apparatus according to claim 3,
wherein the second determination unit determines whether or not the correlation information satisfies each of a plurality of second conditions.

8. The information processing apparatus according to claim 4,
wherein the second determination unit determines whether or not the correlation information satisfies each of a plurality of second conditions.

9. The information processing apparatus according to claim 5,
wherein the second determination unit determines whether or not the correlation information satisfies each of a plurality of second conditions.

10. The information processing apparatus according to claim 2,
wherein the second condition is determined based on a statistical value calculated based on previous pieces of the correlation information of the plurality of participants.

11. The information processing apparatus according to claim 3,
wherein the second condition is determined based on a statistical value calculated based on previous pieces of the correlation information of the plurality of participants.

12. The information processing apparatus according to claim 4,
wherein the second condition is determined based on a statistical value calculated based on previous pieces of the correlation information of the plurality of participants.

13. The information processing apparatus according to claim 5,
wherein the second condition is determined based on a statistical value calculated based on previous pieces of the correlation information of the plurality of participants.

14. The information processing apparatus according to claim 6,
wherein the second condition is determined based on a statistical value calculated based on previous pieces of the correlation information of the plurality of participants.

15. The information processing apparatus according to claim 7,
wherein the second condition is determined based on a statistical value calculated based on previous pieces of the correlation information of the plurality of participants.

16. The information processing apparatus according to claim 8,
wherein the second condition is determined based on a statistical value calculated based on previous pieces of the correlation information of the plurality of participants.

17. The information processing apparatus according to claim 2,
wherein the second condition is determined based on a statistical value calculated based on previous correlation information of each of the participants, for each participant.

18. The information processing apparatus according to claim 1,
wherein the generation unit generates correlation information indicating a correlation between the activity degree and environmental information indicating a surrounding environment of the participant, instead of or in addition to the motion information.

19. An information processing apparatus comprising:
a specifying unit that specifies an activity degree of a participant based on biometric information obtained from a living body of the participant participating in a gathering;
an acquisition unit that acquires motion information indicating a motion of the participant;
a first determination unit that determines whether or not motion information of each of a plurality of participants participating in the gathering satisfies a predetermined third condition;
a second determination unit that determines whether or not the activity degree of the participant satisfies a fourth condition when the motion information satisfies the third condition; and
a notification unit that performs a notification of state information indicating a state of the gathering by using a determination result of the second determination unit.

20. The information processing apparatus according to claim 19, further comprising:
a generation unit that generates correlation information indicating a correlation between the motion information and the activity degree,
wherein the second determination unit determines whether or not the correlation information satisfies the fourth condition.
